(19) United States
(12) Patent Application Publication
(10) Pub. No.: US 2013/0066249 A1
(43) Pub. Date: Mar. 14, 2013

(54) Foot Arch Support for Barefoot Athletes

(71) Applicant: Joshua Paul Eldridge, Duval, WA (US)

(72) Inventor: Joshua Paul Eldridge, Duval, WA (US)

(21) Appl. No.: 13/229,771

(22) Filed: Sep. 12, 2011

(51) Int. Cl. A61F 5/00

(52) U.S. Cl. 602/28

(57) ABSTRACT

An arch support used by an athlete practicing his or her sport in bare feet, which consists of a metatarsal elastic compressive strap and an elastic calcaneal strap originating and terminating on the metatarsal strap using a crossing pattern on the plantar surface of the foot. Design is minimal in structure allowing the athlete to maximize contact of plantar surface of foot with the floor, or other surface, while maintaining support of foot structure.

### BACKGROUND

Arches are the critical first step in the kinetic chain of the human body. If the arches are injured or do not support the foot because of hereditary reasons, the human kinetic chain can have dysfunction including feet, ankles, shins, knees, thighs, hips, lower back, mid back, and neck.

Children athletes that practice their sport in bare feet can be at more risk for these injuries than their counterparts that wear shoes because they have no outside support to support their arches (i.e. orthotics or insole support). Also, major barefoot sports like gymnastics can put enormous amounts of pressure when landing on feet. Studies have shown gymnasts performing more technical landings can have up to 14 times their body weight being exerted on their bodies. The final pressure of this increased body weight will be exerted on the feet and the arches.

One disease in particular effecting young gymnasts is Sever's Disease (calcaneal apophysitis). Sever's is a growth plate injury in which the Achille's Tendon puts excessive pressure on the posterior calcaneal apophysis (growth plate), causing inflammation and irritation of the apophysis. This can create great pain for young athletes. Gymnasts are also more prone to this disease because of the amount of plantar flexion (toe pointing) they do to achieve proper gymnastics foot position. This, along with jumping, landing from heights well over ten feet, and doing all of this barefoot causes extreme pressure on the calcaneal apophysis.

The Foot, Volume 17, Issue 4, December 2007, Pages 178-183 states that taping the arch can be a very effective method in relieving symptoms in individuals with Sever's Disease. The problem is taping requires time and expertise that might not be available for young athletes at their athletic venue. Also, the use of tape becomes exceedingly expensive if taping is done over a lifetime of sport in order to protect the athlete's foot arches.

### DETAILED DESCRIPTION OF THE INVENTION

### Summary of the Invention

This arch support, among other things, allows an athlete to practice their sport barefoot while protecting their arch with very minimal interference from a brace. Braces that have the potential to interfere with the athlete during their sport can be very dangerous and limit what an athlete can accomplish. The design of this invention is to minimize interference while maximizing support of the arch for the barefoot athlete.

This arch support is intended for athletes who practice their sport in bare foot. This invention is not intended to be used with shoes. Barefoot athletes can put enormous amounts of stress on their feet and this invention is intended to help decrease the forces placed on an athlete's arch while they are without shoes.

### BRIEF DESCRIPTION OF THE DRAWINGS

FIG. 1 is a top view of an illustrative embodiment of a barefoot athlete arch support

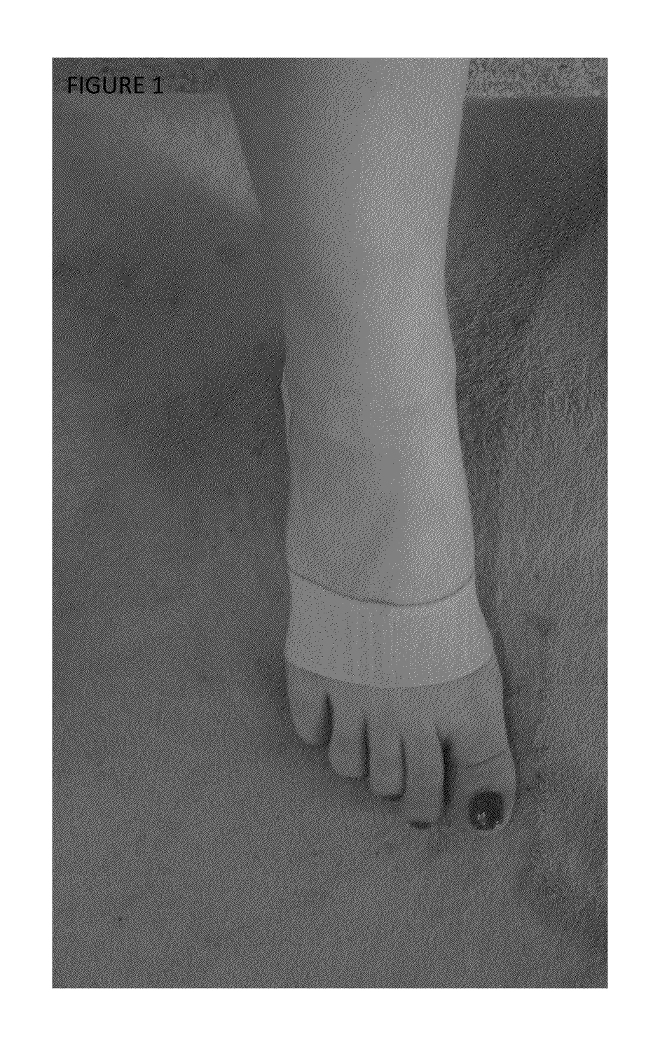

FIG. 2 is a medial perspective view of the foot stabilizer of FIG. 1.

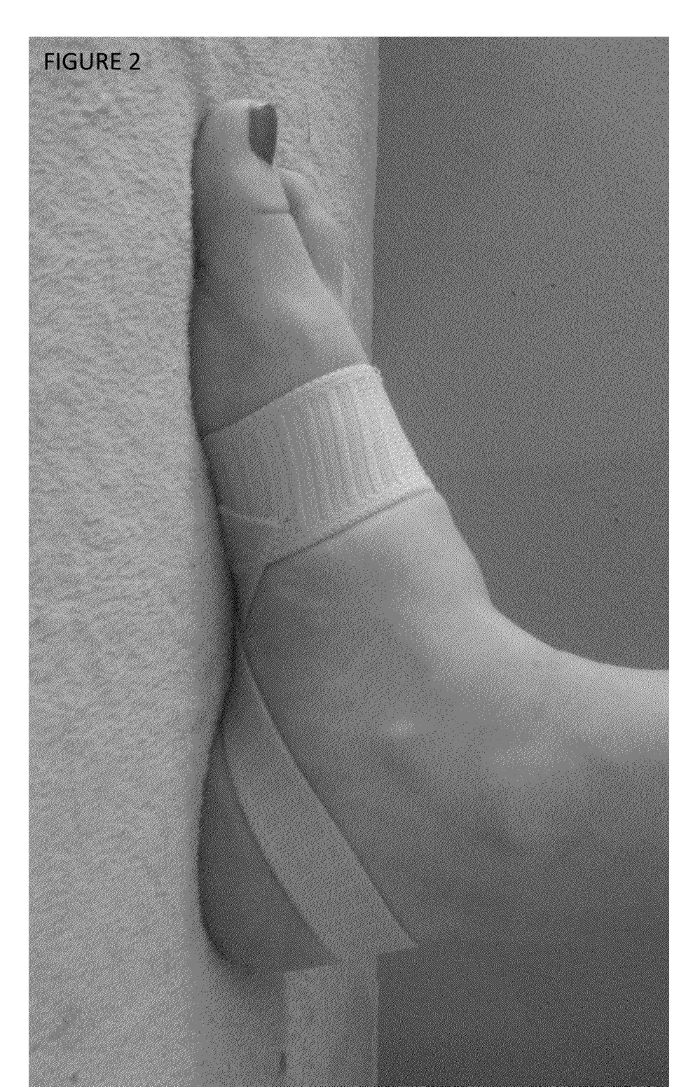

FIG. 3 is a bottom view of the foot stabilizer of FIG. 1.

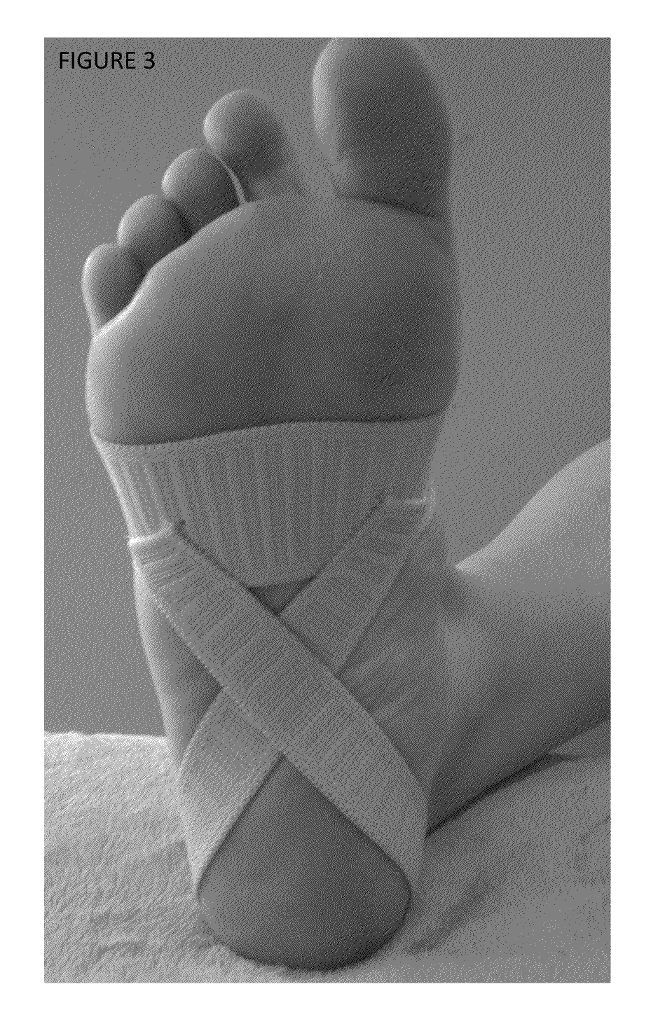

FIG. 4 is a lateral side view of the foot stabilizer of FIG. 1.

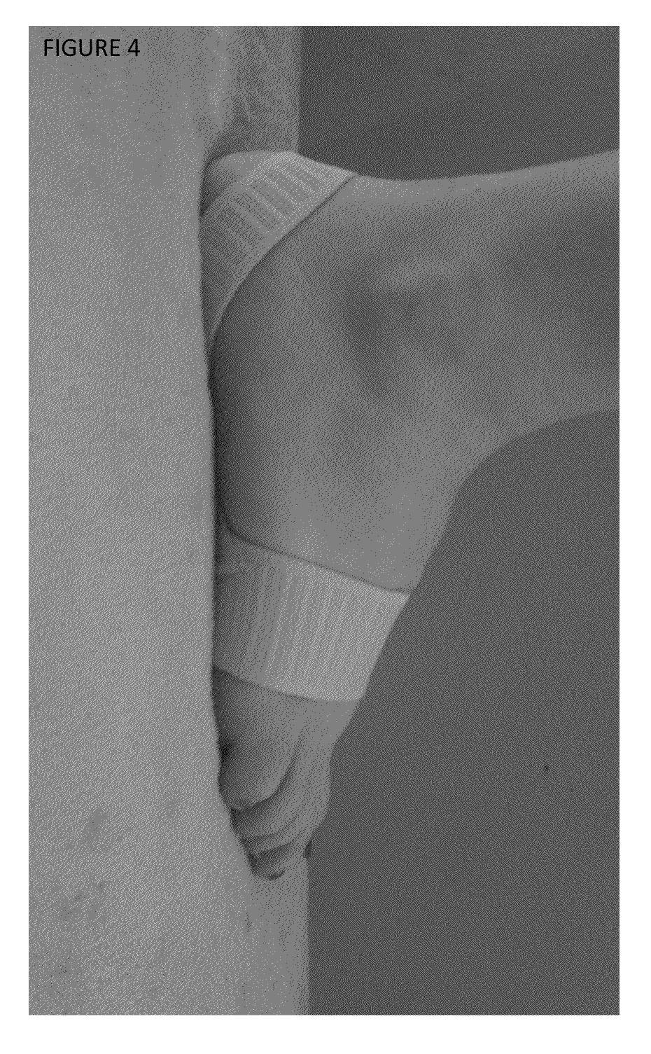

We claim:

1. An arch support consisting of an elastic strap encompassing the forefoot, which accentuates both the transverse arch and the medial longitudinal arch, and a calcaneal strap which originates on the medial portion of the forefoot strap, traverses posteriorly and laterally, around calcaneal tuberosity, and terminates by traversing anteriorly and laterally and attaching to the lateral portion of the forefoot strap, accentuating the medial longitudinal and lateral longitudinal arches.

2. The arch support in claim 1, wherein the elastic strap encompassing the forefoot is overlapped and secured by stitching or sewing, or the same strap can be fastened using hook-and-loop fastener.

3. The hook-and-loop fastener in claim 2, wherein the hook-and-loop fastener must be strong enough so arch support in claim 1 is not compromised during activity causing the arch support in claim 1 to fall off or not fully support arches.

4. The arch support in claim 1, wherein the elastic calcaneal strap is fastened to the forefoot strap both laterally and medially by stitching or sewing.

5. The attachment of the forefoot strap in claim 4 will be fastened at the lateral and medial portions of the forefoot strap at natural angles so they lie to the lateral and medial aspects of the plantar aspect of the foot when the athlete wears the arch support in claim 1.

6. The arch support in claim 1, wherein the arch support will vary in size based on the length and width of the wearers foot.

7. The arch support in claim 1, wherein the color of the arch support is not limited to basic colors but can be varied based on athlete skin color or outfit color.

8. The arch support in claim 1, wherein the elastic band encompassing the forefoot must not be too large to interfere with foot contact with the floor, but not too small to cause the strap to not have enough strength to support said foot arches.

9. The arch support in claim 1, wherein the elastic band that traverses the calcaneus must not be too large to interfere with the fat pad of the foot contacting the floor, but must not be too small to cause the strap to not have enough strength to support said foot arches.
